(19) United States
(12) Patent Application Publication
(10) Pub. No.: US 2022/0338761 A1
(43) Pub. Date: Oct. 27, 2022

(54) Remote Training and Practicing Apparatus and System for Upper-Limb Rehabilitation

(71) Applicant: TACTILE ROBOTICS LTD., Winnipeg (CA)

(72) Inventors: Ali Maddahi, Winnipeg (CA); Mohamed-Amine Choukou, Winnipeg (CA); Amir Mahdi Nassiri, Winnipeg (CA); Yaser Maddahi, Winnipeg (CA)

(21) Appl. No.: 17/726,868

(22) Filed: Apr. 22, 2022

Related U.S. Application Data
(60) Provisional application No. 63/178,735, filed on Apr. 23, 2021.

(51) Int. Cl. A61B 5/11; A61B 5/00; G16H 20/30; G16H 40/67

(52) U.S. Cl. CPC A61B 5/1125; A61B 5/6826; G16H 20/30; G16H 40/67; A61B 2505/09; A61B 2562/0219

(57) ABSTRACT

A rehabilitation system includes left-hand and right-hand rehabilitation apparatuses that cooperate with programming instructions operating on a patient computer and on a therapist computer that is remote from the patient computer. Each apparatus includes a hand section with movement sensors supported on the backside of the hand of the patient from which patient hand movements can be derived, finger sections moveable relative to the hand section with sensors to track flexing and movement of the finger sections relative to the hand, a wrist section supporting vital sign sensors communicating with the patient, and adjustable resistive elements applied to the finger or hand movements. The system enables a therapist to communicate target movements to the user, as well as allowing the comparison between patient movements conducted at different times or with different hands. Sensor data can be used to measure patient performance that is provided to the patient and the therapist.

This application claims the benefit under 35 U.S.C. 119(e) of U.S. provisional application Ser. No. 63/178,735, filed Apr. 23, 2021.

### FIELD OF THE INVENTION

The present invention relates to rehabilitation apparatuses with embedded sensors used by a patient to conduct upper-limb rehabilitation tasks, and more particularly the present invention relates to a training and practicing system and method of use of the training and practicing system such that sensors of the rehabilitation apparatuses capture operating characteristics relating to execution of the rehabilitation exercise by the patient using the rehabilitation apparatuses for subsequent comparison to corresponding operating characteristics of a target movement that the patient attempts to perform so that performance of the patient can be quantitively evaluated.

### BACKGROUND

Impaired hand function is one of the most frequently persisting consequences of stroke, according to Lai S M, Studenski S, Duncan P W, Perera S. Persisting consequences of stroke measured by the Stroke Impact Scale. Stroke. 2002; 33(7):1840-4. Although after stroke, patients appear to benefit from substantial time spent in practice, studies show that they may not be practicing enough as highlighted by Kwakkel G. Impact of the intensity of practice after stroke: issues for consideration. Disability and rehabilitation. 2006; 28(13-14):823-30, and Wolf S L, Winstein C J, Miller J P, Taub E, Uswatte G, Morris D, et al. Effect of constraint-induced movement therapy on upper extremity function 3 to 9 months after stroke: the EXCITE randomized clinical trial. JAMA. 2006; 296(17):2095-104. Compared with other patient populations, patients who have sustained a stroke spend more time alone and inactive or less active in rehabilitation units, more likely because of reduced sensorimotor capacity, according to Bear-Lehman J, Bassile C C, Gillen G. A Comparison of Time Use on an Acute Rehabilitation Unit: Subjects With and Without a Stroke. Physical & Occupational Therapy In Geriatrics. 2001; 20(1):17-27, and Bernhardt J, Dewey H, Thrift A, Donnan G. Inactive and alone: physical activity within the first 14 days of acute stroke unit care. Stroke. 2004; 35(4):1005-9. Thus, there appears to be a practice gap between the amount of training these patients need and the amount they receive.

Therapists who work with patients with stroke use approaches to optimize motor behaviour to restore function and occupational performance. Treatment interventions such as materials-based occupations as disclosed in Lang E M, Nelson D L, Bush M A. Comparison of performance in materials-based occupation, imagery-based occupation, and rote exercise in nursing home residents. Am J Occup Ther. 1992; 46(7):607-11, task-related, as disclosed in Thielman G, Kaminski T, Gentile A M. Rehabilitation of reaching after stroke: comparing 2 training protocols utilizing trunk restraint. Neurorehabil Neural Repair. 2008; 22(6):697-705. 8, and Thielman G T, Dean C M, Gentile A M. Rehabilitation of reaching after stroke: task-related training versus progressive resistive exercise. Archives of physical medicine and rehabilitation. 2004; 85(10):1613-8, or task-specific training, as disclosed in Ada L, Canning C G, Carr J H, Kilbreath S L, Shepherd R B. Chapter 12 Task-Specific Training of Reaching and Manipulation. In: Bennett K M B, Castiello U, editors. Advances in Psychology. 105: North-Holland; 1994. p. 239-65. 10, and Michaelsen S M, Dannenbaum R, Levin M F. Task-specific training with trunk restraint on arm recovery in stroke: randomized control trial. Stroke. 2006; 37(1):186-92, are common methods for remediating impairments and restoring function in the upper limb. These training methods stress the person's active participation, manipulation of goal-oriented tasks or environmental characteristics to drive motor behaviour, and practice of the whole task or components of the task under varying conditions. Patients often know how a movement should be performed but are physically not able to do so, as disclosed in Yang U, Kim G J. Implementation and Evaluation of “Just Follow Me”: An Immersive, VR-Based, Motion-Training System. Presence: Teleoperators and Virtual Environments. 2002; 11(3):304-23. This activity has traditionally been used in athletics in an intuitive manner, to review or reinforce the sequence of movements that make up the action to be performed, as disclosed in Guillot A, Collet C. Construction of the Motor Imagery Integrative Model in Sport: a review and theoretical investigation of motor imagery use. International Review of Sport and Exercise Psychology. 2008; 1(1):31-44. Nilsen D M, Gillen G, Gordon A M. Use of Mental Practice to Improve Upper-Limb Recovery After Stroke: A Systematic Review. American Journal of Occupational Therapy. 2010; 64(5):695-708 has shown that the mental practice has shown to be effective in reducing impairment and improving functional recovery. Nilsen D M, Gillen G, Gordon A M. Use of Mental Practice to Improve Upper-Limb Recovery After Stroke: A Systematic Review. American Journal of Occupational Therapy. 2010; 64(5):695-708 has also demonstrated that mental practice is an effective intervention when added to the physical practice. It is also complex to gauge the dosing required, the most effective protocols, whether improvements are retained, and whether mental practice affects function and perceived occupational performance.

The current state of knowledge in post-stroke hemiparesis neuro-rehabilitation is now ripe for research and therapy practice using technology-assisted interventions. Since stroke incidence is rising, the need has arisen for technology to support the treatment and relieve the therapists' workload. Considering the inequalities in access to care between regions and communities and the occurrence of disasters such as the COVID-19 pandemic, the next step in the field of hand rehabilitation seems to be home-based rehabilitation based on telerehabilitation platform. In addition to enabling access and timely treatment of patients, home-based hand rehabilitation could be an interesting modality of treatment that supports intensity and frequency of practice. Langhorne P, Coupar F, Pollock A. Motor recovery after stroke: a systematic review. Lancet Neurology. 2009; 8(8):741-54 suggests that the higher the intensity and duration of therapy, the better the patient's outcome.

Non-Sensorized Gloves

The utility model discloses a kind of flexible rehabilitation training gloves of stroke patientz, China patent CN206508205U. The model includes flexible expandable finger traction element and driving part; Flexible expandable finger traction element has five and in hand arrangements; All flexible expandable finger traction elements are connected with driving part, and flexible expandable finger traction element stretches under the driving of driving part; Finger bandage and/or finger-stall are equipped with all flexible expandable finger traction elements. The flexible rehabilitation training gloves of the utility model stroke patient, enable stroke patient to carry out rehabilitation training to hand, stimulate re-establishing for nerve pathway, prevent soft tissue adhesion, prevent muscular atrophy, it is to avoid hand tissue ossify because of calcification, so as to promote the rehabilitation of apoplexy illness.

A sports training glove is disclosed in Kobie Landry, Ronald Buyck, inventors. Sports Training Glove. United States patent US20180111035A1. The sports training glove is a garment that covers a person's hand and reduces the hand's ability to apply friction on a graspable object such as a sports ball or other sports equipment. The sports training glove includes a glove body and a friction-reducing membrane. The friction-reducing membrane is integrated across a palm portion of the glove body. The friction reducing membrane may also extend along the finger sleeves and the thumb sleeves of the glove body in order to further reduce the hand's ability to apply friction on a graspable object. Once the sports training glove is taken off of the person's hand, the person should be able to have better control and coordination with the graspable object.

A method for producing exercise-promoting gloves is disclosed in Susumu Ueda, inventors. Method for producing exercise-promoting glove, and exercise-promoting glove. Japan patent JP2006083480A. The description of the patent provides a method for inexpensively producing an exercise-promoting glove having such an unprecedented new technical idea as to guide the movement of the upper limb to promote exercise through controlling bending and stretching and/or inward and outward swinging exercise without imposing so much restriction on the moved areas of finger joints and without passing through any complicated process, and to provide an exercise-promoting glove. The exercise-promoting glove controls the finger joints through fixing joint-controlling bodies each to the back-side surface part and the palm-side surface part of a glove main body corresponding to the vicinity of the thumb pad of the glove's main body, and/or to the back-side phalanx proximalis surface part and the palm-side phalanx proximalis surface part corresponding to the phalanx proximalis, or to the back-side MP joint parts to guide the movement of the upper limb to promote exercise.

A finger rehabilitation treatment apparatus is disclosed in Finger Rehabilitation treatment apparatus. South Korea patent KR20140142895A. An aspect of the present invention provides a finger rehabilitation treatment apparatus, which improves finger's fit by conveniently bending finger joints and can precisely detect the bending angle of finger joints. The finger rehabilitation treatment apparatus, according to an embodiment of the present invention, comprises a base unit, and a finger wearing unit formed to be extended from the base unit and mounting multiple fingers of a user, wherein the finger wearing unit comprises: multiple finger bending sensing units formed to be extended from the base unit to correspond to fingers of a user and detecting the user finger bending angle while bent or spread in a coupled state with fingers of the user; and a first finger sliding fixation unit positioned at a first part of the multiple finger bending sensing units to be fixated to correspond to a first joint at the rear end of the fingers of the user, and sliding along the finger bending sensing unit by the bending extent of the fingers of the user.

Sensorized Gloves

An exercising sensorized apparatus is disclosed in System and method for providing indirect motion feedback during sensorimotor performance rehabilitation and enhancement. China patent CN111246829A. The apparatus is a rehabilitation feedback system for use during the rehabilitation of one or more body parts of a user. The system includes four finger tracking elements and a thumb tracking element supported for movement relative to each other to allow a user to perform a gripping action when the elements are coupled to the user's fingers and thumb. The biasing member provides a resistance for urging each tracking element towards its starting position. The visual barrier is adapted to hide a user's hand at the first side of the visual barrier from the direct line of sight of the user at the second side of the visual barrier. A sensor at the first side of the visual barrier detects the movement of the tracking element and communicates with an indicator element that is perceptible to a user from a second side of the visual barrier.

A finger exercising apparatus is disclosed in Finger exercising apparatus and method for assisting exercise of finger. South Korea patent KR20120133321A. The system gives a motive for rehabilitation exercise. The system is made of a finger rehabilitation exercise device comprised of rehabilitation exercise gloves (10) and a communication terminal (100) which communicates with the gloves. The rehabilitation exercise gloves comprise a wireless communication module (12), a sensor (14) that senses hand operation information, and a control unit (16) which controls to transmit hand operation information sensed by the sensor to the communication terminal. (10) Rehabilitation exercise gloves; (100) Communication terminal; (12) Wireless communication module; (14) Sensor; (16) Control unit

An apparatus for rehabilitation exercise is disclosed in Apparatus for rehabilitation exercise, wearable communication apparatus, and application system and method for applying same. WIPO (PCT) patent WO2012165882A2. Provided is an apparatus for finger rehabilitation exercise, comprising: a rehabilitation exercise glove, which is made in a glove shape, comprising a wireless communication module, a sensor for sensing hand movement information in accordance with at least one of a finger movement, and a change in hand shape, a change in hand position, and a control portion for controlling so that the hand movement information of a user that is sensed by the sensor portion, which is transmitted to a remotely located communication terminal, through the wireless communication module; and the communication terminal comprising a communication module, an object generation portion for generating a virtual hand-shaped object which corresponds to the hand movement information of the user which is received, when the hand movement information is received from the rehabilitation exercise glove by means of the communication module, and a control portion for controlling so that the hand-shaped object that is generated by the object generation portion is displayed through a display screen.

A finger movement measuring apparatus is disclosed in Finger Movement Measuring Apparatus. South Korea Patent KR20160001932A. According to the present invention, a finger movement measuring apparatus comprises: a wearing unit made of an elastic synthetic resin material, tightly worn on the back of a hand and the back of a finger, and provided with a back-of-hand wearing part and a finger wearing part to be elastically deformed by a motion of the back of the hand and the finger; a movement measuring unit which is mounted on an upper portion of the wearing unit, and detects a motion of the finger to measure the movement of the finger. The movement measuring unit comprises: a housing mounted on the back-of-hand wearing part; a control unit disposed in the housing; and a finger movement detection part comprising a first sensor part and a second sensor part mounted on the finger wearing part and positioned on the first segment and the second segment of the finger to detect a motion of each finger segment, and a flexible cable formed lengthwise along the finer to supply power to the first and second sensors to measure a bending movement of the finger.

An apparatus for assisting finger movement is disclosed in Apparatus for assisting finger movement. South Korea patent KR20190127623A. Provided is a finger motion assistance device that provides an elastic force for unfolding fingers while controlling finger motions according to a pulling degree of a wire so as to help a patient who has difficulty in finger motions because of lacking finger muscular force or abnormality of nerve systems to move fingers like a person who has no such problem. The finger motion assistance device comprises: a wearing unit; a first cover extended from the wearing unit to one side thereof; first and second wires towing the first cover; a pulley having the first and second wires wound therearound, respectively; and a motor rotating the pulley. The first and second wires are placed in the direction where the first cover is extended from the first point of the first cover to a second point of the first cover and are placed along at least part of the circumference of the first cover in a mutual opposite direction at the second point of the first cover.

A finger movement measuring apparatus is disclosed in Finger Movement Measuring Apparatus. South Korea patent KR20200024180A. According to the present invention, a finger movement measurement apparatus comprises: a wearing unit made of a synthetic resin material having elasticity, worn to be in close contact with the back of the hand and the back of the fingers, and having a hand back wearing unit and a finger wearing unit to be elastically deformed according to the movements of the back of the hand and the fingers; and a movement measurement unit coupled to an upper portion of the wearing unit and detecting the movement of the fingers to measure finger movement. The movement measurement unit includes: a housing coupled to the hand back wearing unit; a control unit provided inside the housing; and a finger movement detection unit coupled to an upper portion of the finger wearing unit and including a first sensor unit and a second sensor unit which are positioned on the first and second joints of the finger to detect the movement of each finger joint and a flexible cable which is formed long along the finger to supply power to the first and second sensors, so as to measure the bending motion of the finger. Therefore, the finger movement measurement apparatus can measure more precisely the bending motion of finger joints.

A wearing unit for finger movement measuring apparatus is disclosed in Wearing Unit For Finger Movement Measuring Apparatus. South Korea patent KR20160001933A. According to the present invention, a wearing unit for a finger movement measuring apparatus comprises: a back-of-hand wearing part having a shape corresponding to the surface of the back of a hand; and a finger wearing part which is extended from the back-of-hand wearing part and has a shape corresponding to the fingers of a wearer. The finger wearing part comprises: an elastic stretching part which is disposed on a first segment of each finger, and elastically changes a length thereof according to a bending motion of the finger; a joint wearing part worn between the first segment and a second segment of the finger; and a wearing loop formed downwards to allow the finger to be inserted thereinto. The back-of-hand wearing part and the finger-wearing part are made of an elastic synthetic resin material and integrated.

A hand exoskeleton force feedback system is disclosed in Xiaochi Gu, inventors. Hand exoskeleton force feedback system. U.S. Ser. No. 10/423,227B2. This disclosure includes a force feedback device. The force feedback device may include an exoskeleton configured to receive a hand of a user, a base, a force feedback unit, and a microcontroller. The force feedback unit may be coupled to the base of the exoskeleton and the microcontroller. In some embodiments, the force feedback unit is a direct drive motor system.

Andrew Cohen, Genevieve Dion, Mark Winter, Eric Wait, Michael Koerner, “Wearable devices, wearable robotic devices, gloves, and systems, methods, and computer program products interacting with the same”. U.S. Ser. No. 10/248,200B2 discloses one aspect of the invention that provides a wearable device including: at least one compliant region adapted and configured to be placed over a joint of a subject and at least two flexible but less compliant regions coupled to opposite ends of the compliant region. Another aspect of the invention provides a wearable robotic device, including a wearable device as described herein and at least one actuator adapted and configured to move the flexible but less compliant regions relative to each other.

A haptic device that simulates the rigidity of virtual objects is disclosed in Sean Jason Keller, Tristan Thomas Trutna, Nicholas Roy Corson, Garett Andrew Ochs, Raymond King, Elia Gatti, inventors. Haptic devices that simulate rigidity of virtual objects. U.S. Pat. No. 10,324,530B2. Embodiments relate to a system and a method for providing haptic feedback to a user by controlling an area of a surface of a haptic assembly in touch (directly or indirectly) with a user. The haptic assembly can be actuated such that the surface area of the haptic assembly in contact with a user can be adjusted. An area of the haptic assembly in contact with a user can be changed by modifying the shape of the haptic assembly. Hence, by changing the shape of the haptic assembly, a user touching a virtual object in a virtual space with a particular rigidity can be emulated.

A pressure sensing glove is disclosed in Timothy J. Hardy, Nicholas G. Datyner, inventors. Pressure sensing glove. Unites States patent U.S. Pat. No. 9,301,563B2. A pressure sensing glove in which at least five, preferably at least seven and most preferably at least nine pressure sensors are intrinsic to the glove manufacture, usually sandwiched between layers of the glove. Between five to nine pressure sensors are positioned throughout the glove, and the pressure sensors themselves may be capacitive sensors, piezoelectric sensors, air-filled bladder pressure sensors in communication with hollow tubes further connected to electronic pressure sensors, or any other sensor known in the art.

Actuated (Motorized) Gloves

A strengthening and rehabilitating exercise apparatus is disclosed in Strengthening and rehabilitating exercise apparatus. South Korea patent KR20140143557A. The present invention relates to a hand and finger rehabilitating exercise device comprising: a rehabilitating glove including finger inserting parts where fingers are inserted and a wrist inserting part; a bending and stretching adjusting unit which is installed in a length direction of each finger inserting part of the rehabilitating glove and in a length direction of the wrist and includes tubes and a pump unit for supplying air pressure to the tubes, wherein the tubes are equipped with extending and shrinking units which are extended or shrunken by means of air pressure to bend and stretch the finger inserting parts or wrist inserting part; and a finger rehabilitating detecting unit which detects the movement and motion state of fingers in the finger inserting parts of the rehabilitating glove and on the palm.

A system for stroke rehabilitation using position feedback is disclosed in Eleni KOLTZI, Dimitrios Tzovaras, Ioannis KOSTAVELIS, Paschalis SIDERIDIS, Konstantinos PILIOUNIS, inventors. System and Method for Stroke Rehabilitation Using Position Feedback Based Exoskeleton Control Introduction. United States patent US20190336381A1. An improved dual glove exoskeleton system and method for rehabilitation of stroke victims is provided to increase recovery through optic, neural, and muscular stimulation. The proposed approach employs an algorithm that is configured to determine a degree of dysfunction of certain extremities, and in particular, an upper extremity. During rehabilitation and recovery, the proposed system is designed to monitor a position of a unimpaired limb and allow a patient to attempt a mirrored position in a damaged limb. The system and method then completes the movement in an assisted-control manner. The system detects how each finger responds individually to the treatment and chooses an exercise program that is appropriate under the circumstances to further assist with rehabilitation.

Haptic stimulation apparatus is disclosed in James Quest, Stephen Paul Smith, Adam PAWLAK, Howard Morgan Clarke, Cheryl Diane METCALF, inventors. Haptic stimulation apparatus. European Patent EP3337441B1. The apparatus is particularly designed for the treatment of stroke patients, and to apparatus and methods of use thereof. The apparatus can also be used in simulations, for example training, and in the gaming industry.

An immersive upper limb rehabilitation system is disclosed in Immersive upper limb rehabilitation training system. China patent CN210583038U. The utility model discloses an immersive upper limbs rehabilitation training system, include: the system comprises a double-arm rehabilitation robot, a base, a position tracker, a VR helmet, data force feedback gloves, an upper computer control center and passive compliant end effectors, wherein the double-arm rehabilitation robot is arranged on the base and drives the arms of a patient to move through the two passive compliant end effectors; the vision sensor is used for recording the movement of the upper limb of the patient and controlling the movement of the double-arm rehabilitation robot in a feedback manner; the position tracker is used for acquiring the position and posture information of the arm of the patient in real time and transmitting the information to the upper computer control center; the VR helmet is worn on the head of the patient; the data force feedback glove is worn on the hand of the patient; the passive compliant end effector is respectively worn on the forearm and the upper arm of the patient; and the upper computer control center is used for giving quantitative indexes and controlling the display of a virtual training picture. The utility model discloses security, scientific, taste, systematization and the Datamation of upper limbs rehabilitation training process.

A motion assist apparatus is disclosed in Hiromichi Fujimoto, Go Shirogauchi, Keisuke Ueda, Motoshi Hori, Tatsuro Mizutani, inventors. Motion assist apparatus. United States patent U.S. Pat. No. 8,425,438B2. A motion assist apparatus is provided with an attaching member that has a glove shape with finger portions and a middle hand portion that is attachable to one of hands of a user, a back-side actuator that is freely expanded and contracted, and bridged between a tip of each of the finger portions and the middle hand portion on the back side of the attaching member, and a palm-side actuator that is freely expanded and contracted, and bridged between the tip of each of the finger portions and the middle hand portion on the palm side of the attaching member, and in this structure, the palm-side actuator is provided with a wire unit formed of two wires that are disposed on each of the finger portions on the palm side from the tip of each of the finger portions on the back side to the middle hand portion along the finger portion, passing through two side portions of the finger portion, so that intervals therebetween are made different at respective positions corresponding to joints of the finger portion, and rubber artificial muscle is coupled to the wire unit and is formed on the middle hand portion.

Gloves Connected to Virtual Reality Systems

A digital virtual limb is disclosed in Vincent John Macri, Vincent James, Macri, Paul Zilber, inventors. Digital virtual limb and body interaction. Unites States patent. US20200016363A1. The system provides pre-action training control of non-virtual prostheses, exoskeleton body parts, powered orthotic devices, robots or other motile or audiovisual output devices used to improve therapy, rehabilitation, and in some circumstances to lessen or prevent blood clots.

A virtual reality-based hand rehabilitation system with haptic feedback is disclosed in Virtual reality-based hand rehabilitation system with haptic feedback. South Korea patent KR102162922B1. The system receives real-time motion through a finger movement using a bending sensor and a three-axis movement of a hand through an IMU sensor, and expresses a virtual hand image in the content, and a vibration motor when the object in the image corresponds to the virtual hand image The system relates to a virtual reality-based hand rehabilitation system that enables tactile feedback to perform rehabilitation training by giving tactile feedback through.

A multi-user smart glove for virtual environment-based rehabilitation is disclosed in Mark Sivak, Maureen K. Holden, Constantinos Mavroidis, Avi Bajpai, Caitlyn Bintz, Jason Chrisos, Andrew Clark, Drew Lentz, inventors. United States patent US20120157263A1. A low-cost, virtual environment, rehabilitation system and a glove input device for patients suffering from stroke or other neurological impairments for independent, in-home use, to improve upper extremity motor function, including hand and finger control. The system includes a low-cost input device for tracking arm, hand, and finger movement; an open-source gaming engine, and a processing device. The system is controllable to provide four types of multiple patient/user interactions: competition, cooperation, counter-operative, and mixed.

A pneumatic augmented reality tactile feedback platform is disclosed in Sean Kerr, Theodore STODDARD, Neil DAVE, Alejandro Ramirez, Amery LONG, Bahram SHAFAI, inventors. Pneumatic Augmented Reality Tactile Feedback Platform. United States patent US20170300115A1. The invention provides devices, systems and methods for providing a user with haptic feedback, thus connecting the user with a virtual or remote environment. The device includes a plurality of fluid compartments that can be expanded or collapsed to provide tactile sensations to a user. The device design can provide continuous, dynamic and variable feedback that allows a user to distinguish between different virtual objects. The invention provides enhanced spatial and temporal resolution, hence providing more realistic sensory feedback and allowing for immersion into virtual or augmented reality environments.

Apparatus for assisting in finger motion is disclosed in Byeong Geol PARK, Kyung Hwan Yoo, Young Geun Choi, inventors. Apparatus for assisting in finger motion. United States patent US20190091091A1. Disclosed is a finger-motion assisting apparatus comprising a wearing part, a first cover extending from the wearing part to one side, first and second wires pulling the first cover, a pulley around which the first and second wires are wound, and a motor rotating the pulley. The first and second wires are arranged from a first point to a second point of the first cover along the extension direction of the first cover and arranged along at least part of a periphery of the first cover in opposite directions at the second point of the first cover.

### SUMMARY OF INVENTION

According to one aspect of the present invention there is provided a rehabilitation system for measuring performance of an upper limb rehabilitation exercise of a user relative to a target movement using a computing device, the system comprising:

(i) a rehabilitation apparatus comprising:
a hand section arranged to be secured relative to a backside of a hand of the user;
at least one hand movement sensor operatively connected to the hand section so as to be arranged to sense one or more operating characteristics of the hand section;
a plurality of finger sections arranged to be secured relative to respective fingers of the user and being movable relative to the hand section such that the fingers sections are movable with the fingers of the user relative to the backside of the hand of the user;
finger movement sensors operatively connected to the finger sections so as to be arranged to sense one or more operating characteristics of the finger sections; and
a sensor interface in communication with the at least one hand movement sensor and the finger movement sensors; and

(ii) a computer readable memory storing programming instructions thereon which are executable by the computing device such that the computing device is arranged to:
communicate with the sensor interface to obtain the sensed operating characteristics from the movement sensors during the upper limb rehabilitation exercise by the user; and
compare the sensed operating characteristics from the movement sensors to corresponding stored operating characteristics representative of the target movement.
   According to a further aspect of the present invention there is provided a remote rehabilitation training and practicing system having one or more patient workstations that use data acquired from a therapist workstation. The patient workstation has a practicing tool, a finger sensory system to sense an operating characteristic(s) of the fingers, a hand sensory system while the practicing tool is worn on the patient's hands to conduct a rehabilitation task and a wrist sensory system attached to the practicing tool to measure a set of patient's vital signs. The patient workstation has a practicing tool that includes two left and right practicing apparatuses worn on the left and right hands, respectively. The practicing tool may have a sensory system embedded in the practicing apparatuses to read and measure hand movement of the patient's hands, and a processing unit to analyze the data measured by the sensory system according to the operating characteristic(s) sensed by the sensory system of the patient workstation. Alternatively, the training and practicing tool may be operational to perform said rehabilitation procedure, in which the processing unit compares operating characteristics sensed by the patient's impaired hand to those obtained from the patient's healthy hand or therapist's reference hand. The training and practicing tool may have a video recording system and/or a chat system at the therapist workstation and the patient workstation along with computer software and/or mobile application at either workstation. In this manner, the present invention provides a training and practicing system and method of use of the training and practicing system such that (i) a patient using a practicing tool at a patient workstation conducts rehabilitation tasks prescribed by a therapist, and/or (ii) a therapist using an interactive software at a therapist workstation receives quantitative feedback based on a calculated comparison of a patient's impaired hand performance indicators relative to the patient's healthy hand or therapist's reference hand performance indicators at the patient workstation, whereby patients using the invention are able to understand and observe the technic and motion sensory characteristics of the rehabilitation tasks conducted by themselves in a rehabilitation clinic, at home, or any remote location, and (iii) the training and practicing tool consists of a set of smart gloves that a patient puts on their unimpaired and impaired hands to comparatively monitor the performance of the impaired hand, and (iv) the training and practicing tool consists of a software or application that enables the patient to monitor their performance, watch and videotape their activities while communicates with their therapist(s), and (iv) the training and practicing tool consists of a software or application that enables the therapist to monitor and watch their patients' performance and videotape and share the rehabilitation activities with a patient while communicates with their patients.

The system may be arranged to acquire the stored operating characteristics representative of the target movement from a previous upper limb exercise executed on the rehabilitation apparatus.

Alternatively, the system may be arranged to acquire the stored operating characteristics representative of the target movement from a remote computer device in communication with the computing device over a communication network.

The system may further comprise an auxiliary apparatus which is symmetrical in configuration to the rehabilitation apparatus such that one of the apparatuses is arranged to be worn on a right hand of the user and another of the apparatuses is arranged to be worn on a left hand of the user, each of the auxiliary apparatus and the rehabilitation apparatus having said at least one hand movement sensor, said finger movement sensors and said sensor interface with which the computing device is arranged to communicate when executing the programming instructions stored on the computer readable memory. In this instance, the system may be further arranged to be executed by the computing device such that the computing device is arranged to (i) communicate with the sensor interface of the auxiliary apparatus to acquire said stored operating characteristics representative of the target movement from a previous upper limb exercise executed on the auxiliary apparatus, (ii) communicate with the sensor interface of the rehabilitation apparatus to obtain the sensed operating characteristics from the movement sensors during the upper limb rehabilitation exercise by the user operating the rehabilitation apparatus, and (iii) compare the sensed operating characteristics from the rehabilitation apparatus to the stored operating characteristics from the auxiliary apparatus.

The comparison of the sensed operating characteristics from the movement sensors to corresponding stored operating characteristics representative of the target movement may include (i) a calculation of at least one performance metric representative of the sensed operating characteristics from the movement sensors during the upper limb rehabilitation exercise by the user and (ii) a calculation of a similarity value comprising a measure of similarity between said at least one performance metric and a corresponding performance metric defined by the stored operating characteristics representative of the target movement.

The stored operating characteristics may define a range of motion of the target movement. In this instance, the system may be further arranged to calculate a range of motion relating to the upper limb rehabilitation exercise from the sensed operating characteristics from the movement sensors during the upper limb rehabilitation exercise and compare the calculated range of motion to the range of motion defined by the stored operating characteristics.

When the movement sensors include angular velocity sensors and the stored operating characteristics define a speed of motion of the target movement, the system may be arranged to calculate a speed of motion relating to the upper limb rehabilitation exercise from the sensed operating characteristics from the angular velocity sensors during the upper limb rehabilitation exercise and compare the calculated speed of motion to the speed of motion defined by the stored operating characteristics.

When the movement sensors include angular acceleration sensors and the stored operating characteristics define a motion steadiness of the target movement, the system may be further arranged to calculate a motion steadiness relating to the upper limb rehabilitation exercise from the sensed operating characteristics from the angular acceleration sensors during the upper limb rehabilitation exercise and compare the calculated motion steadiness to the motion steadiness defined by the stored operating characteristics.

When the movement sensors including linear acceleration sensors and the stored operating characteristics define a motion stability of the target movement, the system may be further arranged to calculate a motion stability relating to the upper limb rehabilitation exercise by calculating a derivative of an acceleration acquired from the sensed operating characteristics from the linear acceleration sensors during the upper limb rehabilitation exercise and compare the calculated motion stability to the motion stability defined by the stored operating characteristics.

The system may be further arranged to calculate at least one performance metric from the sensed operating characteristics from the movement sensors during the upper limb rehabilitation exercise by the user such that said at least one performance metric defines (i) a movement of the fingers of the user, (ii) a movement of the hand of the user, and/or (iii) a movement of the wrist of the user.

The system may be further arranged to calculate a performance metric representative of the sensed operating characteristics from the movement sensors during the upper limb rehabilitation exercise by the user and generate a graph illustrating a variation of the performance metric over a duration of the upper limb rehabilitation exercise by the user for display on a display of the computing device.

The system may be further arranged to (i) calculate a position of the finger sections and the hand section throughout the upper limb rehabilitation exercise by the user, and (ii) generate a video comprising images graphically representing the calculated position of the finger sections and the hand section throughout the upper limb rehabilitation exercise by the user.

The system may be further arranged to (i) receive video images from a remote computer device over a communications network and display the video images on a display of the computing device, and (ii) capture video images of the user using a camera of the computing device and communicate the captured video images with the sensed operating characteristics to the remote computer device over the communications network.

The system may further include a plurality of resilient finger members, in which each resilient finger member is arranged to be operatively connected between the hand section and a respective one of the finger sections so as to be flexed with movement of the respective finger section relative to the hand section, and in which at least some of the resilient finger members have a different stiffness relative to one another and being interchangeable with one another to vary a resistance of flexing movement of the finger sections.

When the rehabilitation apparatus further comprises a wrist section arranged to be secured relative to a forearm of the user, the system may further include a plurality of resilient wrist members, in which each resilient wrist member is arranged to be operatively connected between the hand section and the wrist section so as to be flexed with movement of the wrist section relative to the hand section, and in which the resilient wrist members have a different stiffness relative to one another and being interchangeable with one another to vary a resistance of movement of the hand section relative to the wrist section.

The system may further include a glove including a hand portion arranged to extend over the backside of the hand of the user and a plurality of finger portions arranged to be worn on the fingers the user, in which the glove includes a plurality of first connectors arranged to releasably connect the finger portions of the glove to the finger sections of the rehabilitation apparatus. One or more second connectors may also be arranged to releasably support the hand section relative to the hand portion of the glove when the glove is worn on the hand of the user. Preferably the glove is readily separable from the hand section and the finger sections of the rehabilitation apparatus so as to be interchangeable with another glove of identical configuration such that the glove can be disposable.

The rehabilitation apparatus may further comprise one or more vital sign monitoring sensors, for example supported on the wrist section of the apparatus, for communication with the user so as to be arranged to sense a state of one or more essential body functions of the user, such that the computing device can be arranged to compare the sensed state to a threshold to determine a notification condition.

According to a further aspect of the present invention there is provided a method of rehabilitation using the system described above in which the method comprises: (i) supporting the rehabilitation apparatus on the hand of the user; and (ii) performing the upper limb rehabilitation exercise including any one of or all of bending, pinching, flexion, extension, adduction and abduction of the fingers or bending, flexion, extension, adduction and abduction of the wrist.

### BRIEF DESCRIPTION OF THE DRAWINGS

One embodiment of the invention will now be described in conjunction with the accompanying drawings in which:

FIG. 1. shows the overall workflow of the invention which includes both the patient and therapist workstations along with the remote data communication system and a database storage system connecting the stations to one another and storing all videos, messages and sensory data, respectively.

FIG. 2. shows the overview of the smart glove designed for the left hand, including sections for the finger sensory system, hand sensory system and the wrist sensory system along with the tightening straps and thumb section of the glove. For the right-hand smart glove, the design is the same as the left-hand one with minor changes to make it adaptable to the right hand.

FIG. 3. shows detailed parts of the left-hand smart glove containing sensory systems, mechanical parts, electronic circuits, power management and communication systems.

FIG. 4 is a screenshot of a graphical output of the patient software illustrating a task performed by the patient and the information of the task (left picture), the graphical information provided to the patient along with the audio/video information during the performance of the task (middle picture) and the welcome page that include the information of the therapists and the patient (right picture).

FIG. 5 is a screenshot of a graphical output of the session creation platform that allows a therapist to create and upload a video and associated information.

FIG. 6 is a screenshot of a graphical output of the schedule platform designed in the therapist software to be assigned to the patient on weekly basis.

FIG. 7 is a screenshot of a graphical output of the chat platform designed to communicate with the patient in a real-time fashion.

FIG. 8 is a screenshot of a graphical output of the patient's performance results in graphical and video output allowing the therapist to evaluate the performance of the patient.

FIG. 9 is a screenshot of a graphical output of the statistical and numerical results of the patient's performance allowing the therapist to evaluate the performance of the patient.

In the drawings like characters of reference indicate corresponding parts in the different figures.

### DETAILED DESCRIPTION OF INVENTION

The invention relates to a smart telerehabilitation platform for the treatment of stroke patients with an upper-limb disability. The invention comprises a patient workstation and therapist workstation. The platform can connect multiple therapists to multiple patients at the same time.

The patient workstation can be set up at home or in a public place such as a clinic. In each patient workstation, there exist a set of smart gloves, along with a software or mobile application which provides the patients with their treatment plan assigned by their therapist(s). Each patient can wear the smart gloves and connect the sensory systems, embedded into the gloves, to the mobile app or software wirelessly. In the procedure of wearing the smart gloves, the patients may or may not need an assistant according to the level of disability. The smart gloves are designed in a way that allow the patients to attach/detach it to/from gloves using a set of connectors and clicking mechanisms. It includes a set of sensors for measurement of the hand motion characteristics such as accelerations, velocities, jerks, Euler angles and bending angles of the fingers during the performance of the rehabilitation tasks, along with the measurement of some of the patient's vital signs such as SpO2, heart rate and body temperature. Each patient can download the mobile app on their smartphone or the software on their laptop/desktop PC and register an account in the software or application. Once they are registered on the platform, an authentication system will approve their registration and connect them to their therapist(s). In the developed application, each patient has their own personal page, which is not shared with others. Each patient can see the list of rehabilitation tasks assigned by their therapist in application/software. Each task comes with a training video pre-recorded by the therapist along with other information about the task, such as the duration of the task, the number of sessions, sets and repetitions required for the task. The information is given by their therapist(s). Since the platform can be used for remote treatment of the patients, the patients are also given an option of recording the video of their activity during the rehabilitation session and sending the video to their therapist(s). Before starting any rehabilitation task, the patients need to connect the hardware system embedded in the gloves to the mobile application or software, which allows the application or software to capture the sensory data wirelessly. Once the smart gloves are connected to the mobile app or software, the patients are directed to perform a sanity check for the wireless connection and start the rehabilitation task using the provided smart gloves. Patients can also see the results of their performance on the mobile app or software using a set of key performance indices (KPIs) defined in a layperson's language as well as a set of graphical indicators. During the performance of the rehabilitation task, patients are provided with the option of toggling between two videos; one video is the training video recorded by the therapist(s) for each task, and the other video is the real-time taping of the patient's activity using the front-camera of the cellphone or an external camera connected to the laptop or desktop PC in the case of using the software instead of the mobile app. With respect to the set of smart gloves, the patients need to wear the smart gloves before starting each rehabilitation task. Depending upon which hand is impaired, the patients are also asked to wear the smart glove of the other hand (i.e., unimpaired or healthy hand) and perform the assigned rehabilitation task once with the unimpaired hand aiming to capture the sensory data and use them as the reference data for calculation of the KPIs and statistical analysis of the patient's performance. The KPIs include, but not limited to, fingers' range of motion (ROM), fingers' movement speed, fingers' movement steadiness, wrist ROM (including flexion, extension, ulnar deviation, radial deviation, supination and pronation), wrist speed along 3 axes, wrist steadiness and overall stability of the hand motion. Afterwards, the patients can start conducting the same rehabilitation task with the help of the smart glove worn on the impaired hand(s). In case of both hands impaired, the patients need to conduct the rehabilitation task(s) with both hands while the reference data will be provided by their therapist for each specific task. This way, the reference data included in the KPIs are shown to the patients graphically which provides them with a clue on how well they are performing the task. The graphical representations of the KPIs are included in the software or application designed for the patient. The same reference data will also be sent to the therapist for analyzing the patient's performance and their progress over the upper-limb rehabilitation period.

On the therapist workstation, each therapist can register to the software developed for communicating with their patients. Same as the patient's side, an authentication system approves the therapist registration and creates their own personal page in the software. The software can either be a web-based application or a desktop application, or a mobile application. Once the therapist registers in the software, they can log into the software, which then navigates them to their own personal page where they can invite their patients and add the various rehabilitation tasks to the list of tasks. The therapists are provided with the option of recording the video of the task and uploading it along with a description for each task. The recorded video is considered as the training video, which will be sent to the patient workstation upon assigning a task to the patients. The therapist can have access to each patient's information and history of their activities along with other features such as the KPIs related to the tasks they have accomplished. On each patient's page, the therapist can have access to the patient's detailed information and the video and KPIs of the tasks performed by the patients in each training log. In addition, the therapist can write the notes and submit it to the patient remotely. When assigning the rehabilitation task to the patient, the therapist can define the duration required for the specific task along with the number of times that the patient needs to repeat the task, etc. Furthermore, the invention offers an audiovisual platform for remote communication between the patient and therapist, which allows both patient and therapist to talk to one another remotely and share their thoughts on the rehabilitation procedure.

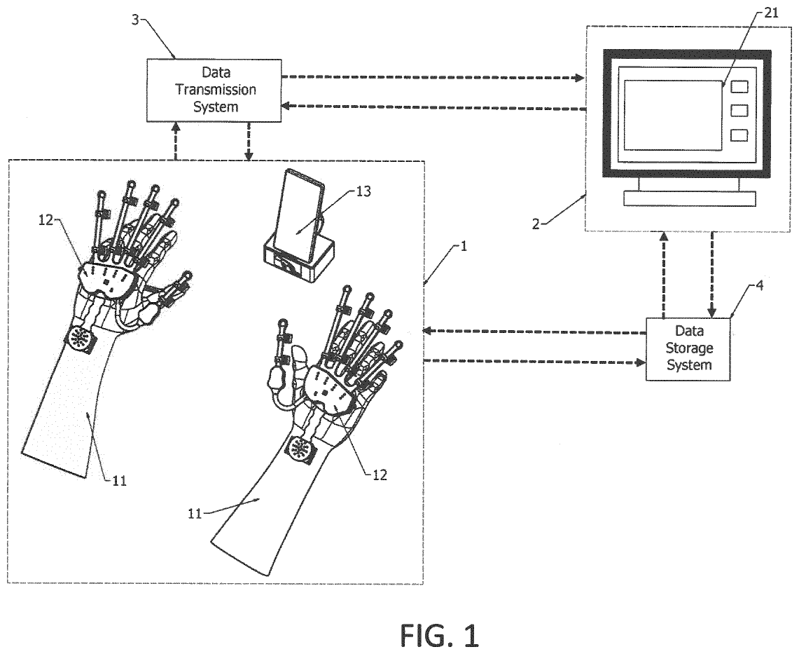

FIG. 1 presents the overall scheme of the invention. Said patient workstation 1 is the location where a patient is situated and can communicate with said therapist workstation 2 via said data transmission system 3. All data transferred between workstations 1 and 2 are stored in the data storage system 4, which can be a local or cloud database service. Said data transmission system 3 is a wireless transmission system that enables both patients and therapists to work with the rehabilitation platform either in the clinic or at home. Said therapist workstation 2 includes a said software 21, which can either be a desktop or web-based or mobile application. Said software 21 is the tool that allows the therapist to define rehabilitation tasks, have access to their own patients, assign the tasks to the patients and observe their patient's performance for evaluation and treatment purposes using a set of KPIs. Said patient workstation 1 has a software 13 which can either be a desktop or web-based or mobile application. The workstation 1 also includes a set of smart gloves 12 worn on either one or both hands 11.

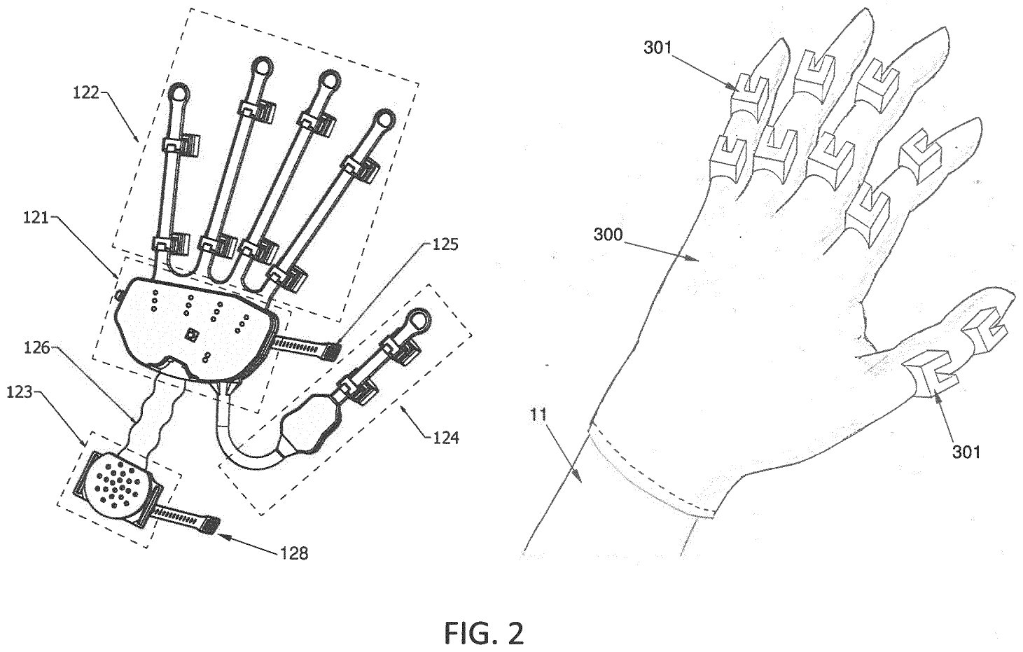

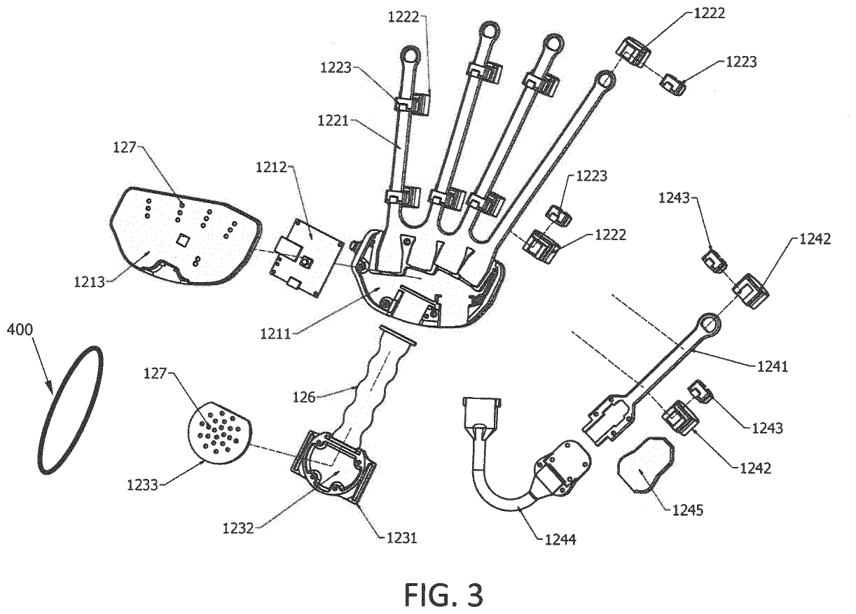

FIG. 2 demonstrates different components that constitute the smart gloves 12. Besides, FIG. 2 provides a schematic of the disposal glove that can worn by patients. Said left-hand and/or right-hand smart glove 12 is worn on patient's hands. Said smart gloves 12 has a set of finger sensory systems located inside the said finger section 122, a hand sensory system inside the said hand section 121 and a wrist sensory system placed inside the said wrist section 123. All sensor data are wirelessly transmitted to said software 13 via Wi-Fi or Bluetooth or Bluetooth Low Energy (BLE) or any other wireless communication protocol. Said wrist section 123 is attached to said hand section 121 via the connection part 126. The wrist section can be tightened to the patient's wrist via said strap 128. There also exists a said strap 125 to tighten the smart gloves around the patient's hand in a comfortable way. Said thumb section 124 of said smart gloves 12 is designed to handle the thumb's motion characteristics. The disposal glove 300 has a set of said connectors 301 to be engaged with said connectors 1223 and 1243 (see FIG. 3) of said smart gloves 12 in case the patient wants to use disposal gloves.

FIG. 3 shows detailed components of various sections of said smart gloves 12. Said hand section 121 include a base 1211 which holds the said main hardware system 1212. Main hardware system 1212 include a processing unit, power management system, data communication module, push button, etc. It also includes a hand sensory system for measuring motion characteristics of the hand with respect to the wrist. Said main hardware 1212 is covered by said enclosure 1213 which also holds a set of said hooks 127. Said hooks 127 can be connected to fingers hooks located on top of said clicking connector 1223 using a set of elastic rubber bands 400, with different stiffnesses, for strengthening the fingers muscles. Said finger section 122 has four elastic bars 1221 which also contain finger sensory system used to measure fingers' motion characteristics. Said elastic bars 1221 are attached to said fingers holders 1222 using a set of said fingers clicking connectors 1223. The said fingers holders 1222 can be in a circular shape to be engaged with the patient's four fingers directly in the case of not using disposal glove 300. If the disposal gloves 300 are used, said finger holders 1222 can be removed from the said smart glove 12 and said connectors 301 attached to disposal glove 300 will be engaged with said clicking connectors 1223. The sensors inside the said four elastic bars 1221 are connected to the main hardware system 1212. Said wrist section 123 also has a base 1231 which is tightened to the patient's wrist using strap 128 and has a hardware system 1232 and a said wrist enclosure 1233 to cover the hardware 1232. The said hardware 1232 includes a wrist sensory system to measure some of the patient's vital signs such as temperature sensor, SpO2, heart rate sensors, a pedometer sensor etc. The mechanical part and wirings of said wrist section 123 are connected to the hand section 121 via the connector 126. Said wrist enclosure 1233 also holds a set of hooks 127, which are similar to the ones formed on top of the hand enclosure 1213. The wrist hooks and hand hooks can be connected to one another using a set of elastic rubber bands 400, with different stiffnesses, for strengthening the wrist muscles and joints. Said thumb section 124 includes an elastic connector 1244 which connects the said thumb elastic bar 1241 to the said hand section 121. The said thumb elastic bar 1241 contains sensors for measuring thumb motion characteristics. Said elastic bar 1241 is attached to said thumb holders 1242 using a set of said thumb clicking connectors 1243. The said thumb holder 1242 can be in a circular shape to be engaged with the patient's thumb directly in the case of not using disposal glove 300. If the disposal gloves 300 are used, said thumb holders 1242 are removed from the said smart glove 12 and said thumb section connectors 301 attached to disposal glove 300 will be engaged with said thumb clicking connectors 1243. Combinations of said connectors 301 and said clicking connectors 1223 and thumb clicking connectors 1243 makes the smart gloves detachable. Said thumb clicking connectors 1243 have also hooks with the similar functionality as the ones located on the said fingers clicking connectors 1223. The thumb housing 1245 is used to cover and locate a part of the electronic parts of the thumb section 124.

As described herein, the present invention relates to a rehabilitation system for measuring performance of an upper limb rehabilitation exercise of a user relative to a target movement. The system overall comprises one or more rehabilitation apparatuses 12 is represented by the smart gloves 12 for the right and left hands as described above and software that is executable on a computing device 13 so that the computing device 13 can interact with the rehabilitation apparatuses 12 to acquire data for performing various functions as described in the following.

The patient workstation 1 at the site of the patient user includes the rehabilitation apparatuses 12, the user computing device 13 and/or the disposal gloves 300. An example device 13 includes a software having computer readable memory therein that stores the programming instructions associated with the present invention thereon such that the instructions can be executed by the processor of the device. The device 13 further includes an integral display for displaying various information to the user as described in the following. The computing device 13 also typically comprises a camera for capturing image data or video relating to the patient user executing the rehabilitation exercise.

The patient workstation 1 communicates with a therapist workstation 2 in which the therapist workstation comprises a separate computer device 21 also having a processor and a computer readable memory storing programming instructions thereon for executing the various functions described in the following. The therapist workstation 2 and the patient workstation 1 communicate with one another over a suitable communications network 3 which may include a local network, a mobile telecommunications network, the Internet, and the like, so that various information can be exchanged between the computing device 13 of the patient user and the computer device 21 used by the therapist. The system may further comprise a remote server functioning as a data storage system 4 which communicates over the communications network with each of the computer device 21 of the therapist and the computing device 13 of the patient user. The remote server further comprises a processor and a computer readable memory storing programming instructions thereon so as to be suitable for executing some of the functions described in the following if desired. Collectively the remote server, the computer device 21 of the therapist and the computing device 13 of the patient, together with internal processors of the rehabilitation apparatuses 12, all function together as an overall controller of the various functions performed by the system of the present invention as described in the following.

Each of the rehabilitation apparatuses 12 is configured either in a right-hand configuration for being fitted to the right hand of the patient user or a left-hand configuration for being fitted to the left-hand of the patient user. The apparatuses 12 are typically provided as a pair including one left-hand configuration and one right-hand configuration to be worn by both hands of the patient is shown in FIG. 1. The right-hand and left-hand configurations of the apparatuses 12 are symmetrical with another, but are otherwise fully identical to one another.

Each rehabilitation apparatus 12 thus generally includes a hand section 121 arranged to be secured generally against the backside of the hand of the user in operation. The hand section 121 includes a housing base 1211 and a housing cover 1213 that collectively define a housing for receiving electronic components 1212 therein. The electronic components comprise a printed circuit board including a processor and a memory storing programming instructions thereon. The printed circuit board further defines a sensor interface thereon which communicates with the various sensors of the fingers, wrist, hand and thumb as described in the following, for in turn communicating all of the sensor data to the computing device 13 for further processing. Some degree of processing of the data to determine one or more operating characteristics of the various sections of each apparatus 12 or to further calculate one or more performance metrics as further described below may occur within the printed circuit board of the hand section 121 as opposed to on the computing device 13. The printed circuit board further defines a plurality of hand movement sensors thereon for sensing velocity, acceleration, angular velocity, and angular acceleration and the like to enable the system to track motion of the hand section as well as tracking position, orientation, speed, and acceleration of linear movement and rotational movement about one, two or three axes as described herein. The movement data of the hand section can be subsequently interpreted by the system as a corresponding movement of the wrist including flexion, extension, ulnar deviation, radial deviation, supination and/or pronation. A strap 125 is coupled externally to the housing which is arranged to be adjustably secured about the hand of the user to secure the hand section 121 against the backside of the hand of the user. The printed circuit board 1212 further includes a power button that is accessible externally through an opening in the housing cover 1213 and a status indicator light that also communicates through an opening in the housing cover 1213 to indicate the operating status of the apparatus 12.

Each apparatus 12 further includes a plurality of finger sections 122 in which one finger section is provided for each of the fingers of the user. Each finger section includes an elongate bar 1221 which is flexible and elastic or resilient to be biased to return to a linear unflexed position as shown in the figures. Each bar 1221 is anchored at its base between the base 1211 and the cover 1213 of the hand section approximately in alignment with a corresponding knuckle of the hand of the user. Each bar extends outwardly to span a length which is greater than the fingers of the user. Each bar 1221 is intended to be flexed into a curved shape to follow the flexing of finger movement of the user. The bar 1221 is coupled to a respective finger by a pair of finger holders 1222 supported on each bar 1221 or to a respective finger by a pair of connectors 301 attached to disposal gloves 300. Each finger holder 1222 or connector 301 is supported on a respective coupler 1223 in which the coupler is supported for longitudinal sliding movement along the bar 1221. Each finger holder 1222 is a loop that receives the finger extending therethrough and which includes a suitable connector formed thereon which is releasably connected to a corresponding connector on the coupler 1223. Two finger holders are positioned at spaced positions along each bar 1221 so that a first coupler can be secured about the finger of the user near the base of the finger and a second coupler can be secured about the finger near the fingertip. The same procedure is applicable when the patient uses the disposal gloves to replace finger holders 1222 by connectors 301. Each finger section 122 is further equipped with a finger movement sensor incorporated into the elongate bar 1221 of the finger section. Each finger sensor is a flexible conductive element having an electrical resistance that varies with increasing flexing and bending stress applied to the sensor element so that the changing resistance is indicative of a flexing angle of the element and the corresponding bar 1221 within which the element is incorporated. The finger sensors are in communication with the printed circuit board in the hand section 121 so that all sensed finger movement data is reported back to the processor within the hand section 121 which in turn relays the data to the computing device 13 using the sensor interface of the printed circuit board.

Each apparatus 12 includes one thumb section 124 which is similar in configuration to each finger section yet includes some distinctions. The thumb section 124 differs from the finger sections 122 in that the thumb section includes a plurality of thumb sensors capable of tracking velocity, acceleration, angular velocity, and angular acceleration linearly and rotationally about one, two or three axes similarly to the sensors of the hand section in which the portion of thumb sensors are situated within the housing 1245. This enables tracking various aspects related to the position and orientation of the thumb section 124 relative to the hand section 121. The thumb housing 1245 is connected to the hand section housing through a resilient connecting bar 1244 that positions the thumb section 124 relative to the hand and finger sections for alignment with the thumb of the user but which readily allows free movement of the thumb relative to the hand.

The thumb section 124 further includes a thumb bar 1241 extending from the thumb housing 1245 so as to be configured similarly to the finger bars 1221 in that the thumb bar is resilient to be biased towards a linear unflexed position yet follows the flexing movement of the thumb during exercise. A pair of thumb holders 1242 are provided as loops which receive the thumb extending therethrough at spaced apart positions in proximity to the base of the thumb and the tip of the thumb similar to the holders 1222 of the finger sections. Also, similarly to the finger sections, each thumb holder 1242 is releasably coupled using suitable connectors to a corresponding coupler 1243 in which the coupler 1243 is mounted for longitudinal sliding along the thumb bar 1241. In case of using disposal glove 300, the thumb holder 1242 is replaced by connectors 301 attached to disposal glove 300. The thumb bar 1241 also includes a thumb sensor in the form of an elongate conductive element incorporated into the thumb bar 1241 with variable resistance dependent upon the flex angle of the thumb bar so that a flex angle of the sum based on a corresponding flexing of the thumb bar can be determined similarly to the finger sensors.

Each apparatus further includes a wrist section 123 including a housing base 1231 and a housing cover 1233 releasably attached to the housing base 1231 to enable a printed circuit board of electronic components 1232 to be received within the housing. A strap 128 is provided for connection to the housing to be releasably secured about the wrist of the user. The printed circuit board 1232 within the wrist section communicates with the printed circuit board of the hand section 121 through a suitable communication connector 126.

The printed circuit board of the wrist section 123 defines one or more vital sign monitoring sensors for communication with the user so as to be arranged to sense a state of one or more essential body functions of the user. The programming instructions are further ranged to compare the sensed states to one or more threshold to determine a notification condition. The vital sign monitoring sensors can be selected from the group consisting of a temperature sensor, a global positioning sensor, an accelerometer sensor, an IMU, one or two acoustic sensors, a gyroscope sensor, a magnetic sensor, a distance sensor, a skin sensor, an ECG sensor, a respiratory rate sensor, a microphone for cough detection, a mobility sensor, a step count sensor, a Bluetooth module, a Bluetooth low energy sensor (and/or Wi-Fi) for detecting social distancing among different wearers and for data transmission, and combinations thereof.

Each apparatus 12 may be further arranged so that resistance can be provided in an adjustable manner to the various movements of the finger sections relative to the hand section, the thumb section relative to the hand section, or the hand section relative to the wrist section. In this instance, a plurality of resilient members 400 is provided in which some of the resilient members are identical in configuration to one another but different in resistance or stiffness so that the amount of force required to resiliently deform the resilient members varies and thus the amount of resistance applied to the flexing movement by the user will also vary.

Each resilient member is a continuous loop or band so as to be arranged for alignment and releasable connection to hooks formed on the connectors 1223 of each finger section and/or on the thumb connectors 1243 and to corresponding hooks 127 on the hand section such that each resilient member 400 is operatively connected between the hand section and a corresponding one of the finger/thumb sections. The resilient members 400 are provided in different sizes for matching the different fingers respectively in which all of the resilient members associated with a particular finger are interchangeable with one another and have different stiffness or resilience relative to one another.

The similar resilient members 400 can be arranged for alignment and releasable connection to hooks 127 on the hand section and corresponding hooks 127 on the wrist section such that each resilient member 400 is arranged to be operatively connected between the wrist section in the hand section for resilient distortion when the hand is moved relative to the forearm corresponding to a wrist movement.

As further shown in FIG. 2, the loops 1242 of the thumb holders and the loops 1222 of the finger holders can be interchanged with the glove 300 arranged to be worn on the hand of the user and which includes integral connectors 301 formed thereon which mate with the couplers 1223 of the finger sections and the couplers 1243 of the thumb section. As illustrated, the glove 300 includes a hand portion arranged to extend over the backside of the hand of the user, a plurality of finger portions arranged to receive the fingers of the user therein, and a thumb portion arranged to receive the thumb of the user therein. When worn on the hand of the user, the integral connectors 301 are aligned with the ideal positioning of the couplers 1223 of the finger sections and the couplers 1243 of the thumb section to form a releasable connection between the couplers 1223/1243 in the integral connectors 301 on the finger and thumb portions of the glove. The hand section 121 can again be secured relative to the hand using a strap 125 that is secured about the and portion of the glove, or alternatively an arrangement of integral connectors on the base of the hand section 121 and the backside of the hand portion of the glove can releasably cooperate with one another to mount the hand section to the glove. The wrist section remains secured about the forearm or wrist area of the user using the strap 128 as described above to maintain communication of the vital sign sensors with the skin of the patient user. When the finger sections, the hand section, the thumb section and the wrist section are separated from the glove 300 the remaining components of the glove include low-cost components of a plastic or cloth arrangement of a hand portion and finger portions with the integral connectors 301 that may also comprise a plastic material. The glove 300 conduct be treated as a disposable component of the apparatus 12 so that a different glove 300 is used with each patient user when multiple patient users make use of the same apparatus 12 for example.

In use, the patient user will typically perform an exercise by first putting on one of the apparatuses 12 and then initiating the rehabilitation exercise which may include various movements including bending, pinching, flexion, extension, adduction, abduction and the like. The system captures various data output by the sensors during the exercise performed by the patient user. As the data is recorded in real-time, or subsequent to the exercise, the system defines one or more operating characteristics relating to the movement performed by the user and subsequently calculates one or more performance metrics. The operating characteristics and/or the calculated performance metrics are compared to corresponding operating characteristics or calculated performance metrics that define a target movement that the user is attempting to perform in performing the rehabilitation exercise.

The operating characteristics and performance metrics defining the target movement are stored on the system and can be obtained by various means. In some instances, the target operating characteristics or performance metrics are dictated by the system as goals which are desirably obtained. Alternatively, the defined target movement can be prescribed by a therapist through their computer device 21 for subsequent communication to the computing device 13 of the patient user for subsequent comparison after the user performs the rehabilitation exercise. In yet a further instance, the stored target movement may instead merely comprise operating characteristics or metrics which were captured by the user's previous attempt to perform the same rehabilitation exercise so that the user can gauge their improvement from one exercise session to the next. In yet a further instance, when provided with two apparatuses including one right-hand configuration apparatus and one left hand configuration apparatus and when the user is rehabilitating only one hand, the user performs the rehabilitation exercise using the other hand which is not impaired so that the corresponding apparatus 12 of the unimpaired hand is used to capture operating characteristics and calculate performance metrics relating to the rehabilitation exercise performed by the unimpaired hand. This data relating to the movement by the unimpaired hand is stored by the system as a target movement which the user then attempts to mimic with the impaired hand using the other rehabilitation apparatus 12.

In each instance, the comparison of data may include the calculation of a similarity value that represents an overall similarity between the data acquired during the rehabilitation exercise and the data corresponding to the target movement to which the rehabilitation exercise is being compared. In one example, the similarity value is calculated by (i) determining a first value corresponding to one or more operating characteristics or one or more performance metrics relating to the target movement, (ii) determining a second value corresponding to one or more operating characteristics or one or more performance metrics relating to the performed rehabilitation exercise, (iii) calculating a difference between the first value and the second value, (iv) dividing the calculated difference by the first value, and (v) optionally multiplying by 100 to express the calculated result as a percentage.

The following list are examples of operating characteristics captured by the various sensors and the resulting performance metric or KPI that is calculated based on the captured operating characteristics:

Each of the sensing elements incorporated into the finger bars 1221 and the thumb bar 1241 is capable of measuring a flex angle of the corresponding finger or thumb so that minimum and maximum flex angles can be determined as operational characteristics which are then used to determine an overall range of movement as a performance metric for each individual finger section or thumb section of the apparatus 12. In addition, movement sensors capable of measuring or being used as a basis for calculating velocity, acceleration, angular velocity and/or angular acceleration are also associated with each of the finger sections in the thumb section. In this instance, average angular velocities throughout the range of motion of each finger and thumb represent operational characteristics that can be used to calculate a performance metric comprising an overall speed of movement of each finger and thumb. Furthermore, average angular accelerations throughout the range of motion for each thumb and finger represent operational characteristics that can be used to calculate a performance metric comprising an overall steadiness for each thumb or finger.

The additional movement sensors within the hand section 121 are capable of measuring or being used as a basis for calculating velocity, acceleration, angular velocity and/or angular acceleration relative to one, two or three axes. In this manner performance metrics for range of movement, speed and steadiness can be determined for six wrists movements corresponding to flexion, extension, ulnar deviation, radial deviation, supination and pronation. Specifically operational characteristics such as minimum and maximum values can be used as input for calculating the overall range of motion as a performance metric for each of the identified wrist movements. In addition, operational characteristics such as average angular velocity values relating to pitch, yaw, and roll angles can be used as input for calculating the speed of movement as a performance metric for each of the identified wrist movements. Furthermore, operational characteristics such as average angular acceleration values can be used as input for calculating the steadiness of movement as a performance metric for each of the identified wrist movements.

Finally, operational characteristics such as average values for various obtained accelerations can be used as inputs for calculating a derivative to measure jerk magnitude, which in turn defines overall stability of the hand as a performance metric.

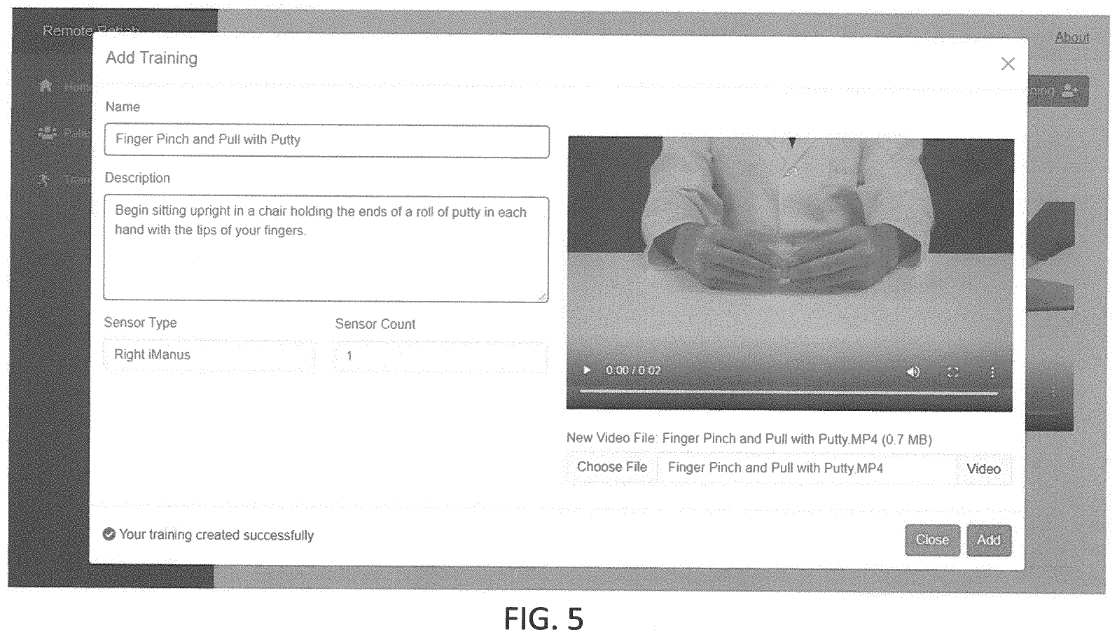

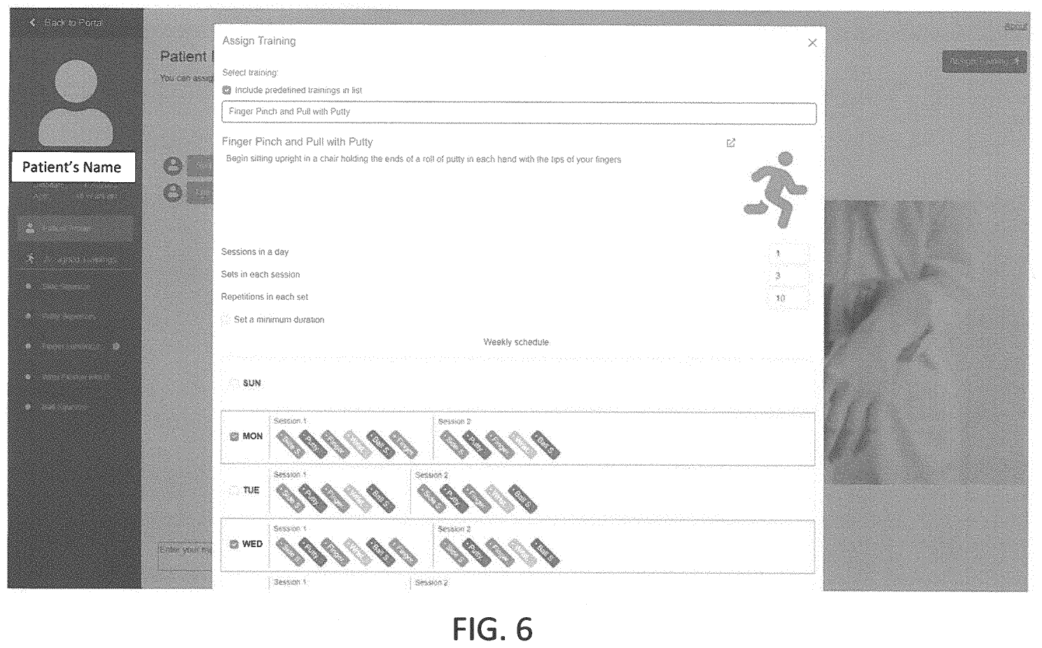

In practice, the system according to the present invention allows ready communication between a therapist and a patient user in addition to measuring performance of rehabilitation exercises by the patient user. The software on the computer device 21 of the therapist provides the therapist with an interface that summarizes a rehabilitation program for each one of multiple patients in which each patient is provided with their own pair of apparatuses 12 and an operating software on their own computing device 13 as described above. For each patient, the therapist using the computer device 21 can select an exercise program directing the patient to perform specific exercises according to a schedule as represented in FIG. 6. For each exercise, the therapist can select instructions to be included such as a demonstration video according to FIG. 5 and special instructions attached in corresponding text boxes that can be customized by the therapist for subsequent display to a patient on their own device. The schedule, and details of assigned tasks can all be communicated from the computer device 21 of the therapist to the computing device 13 of the user.

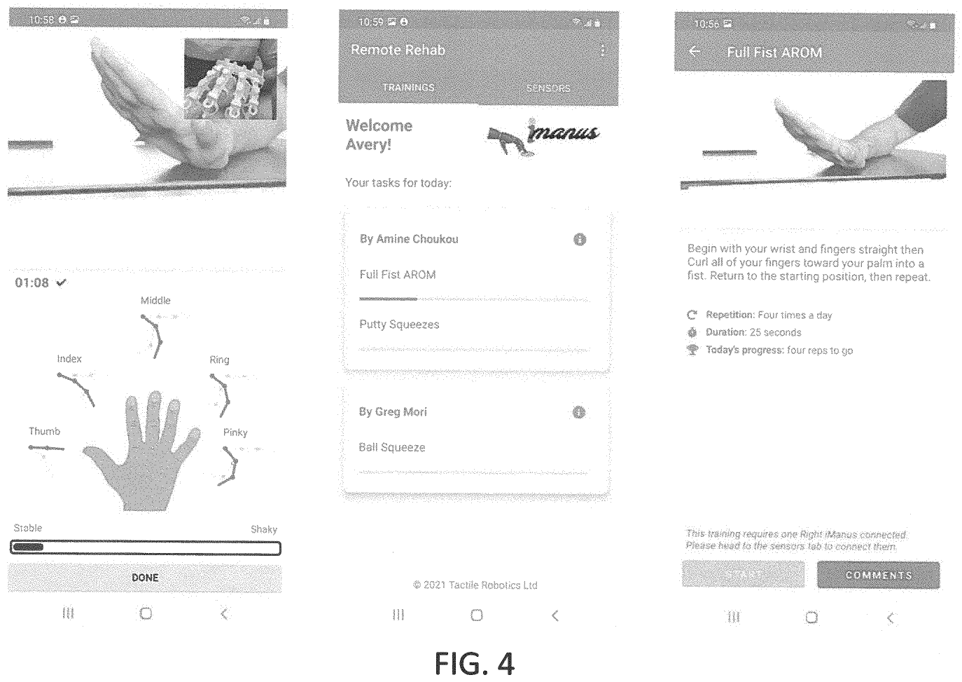

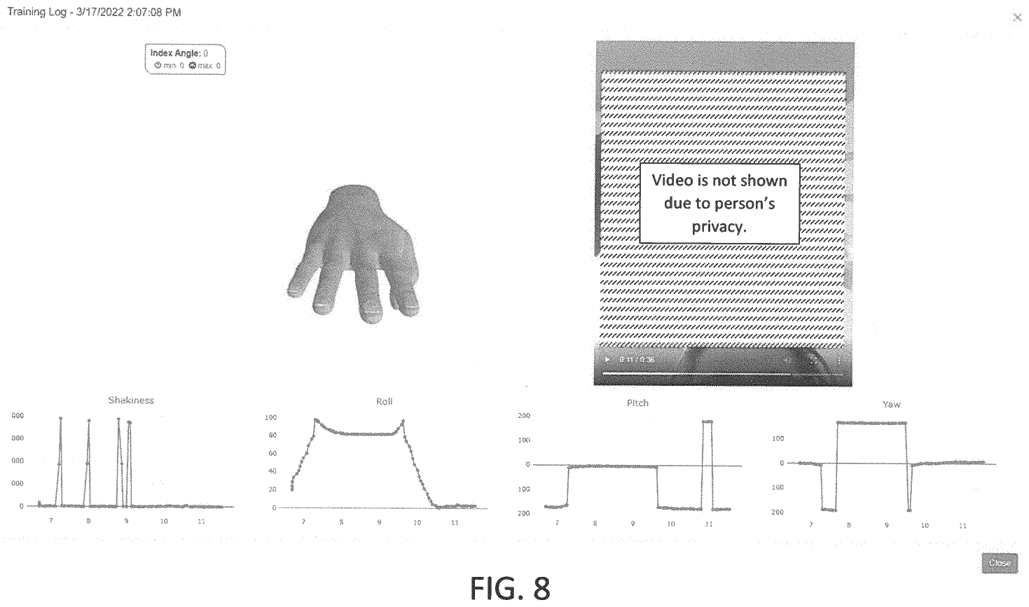

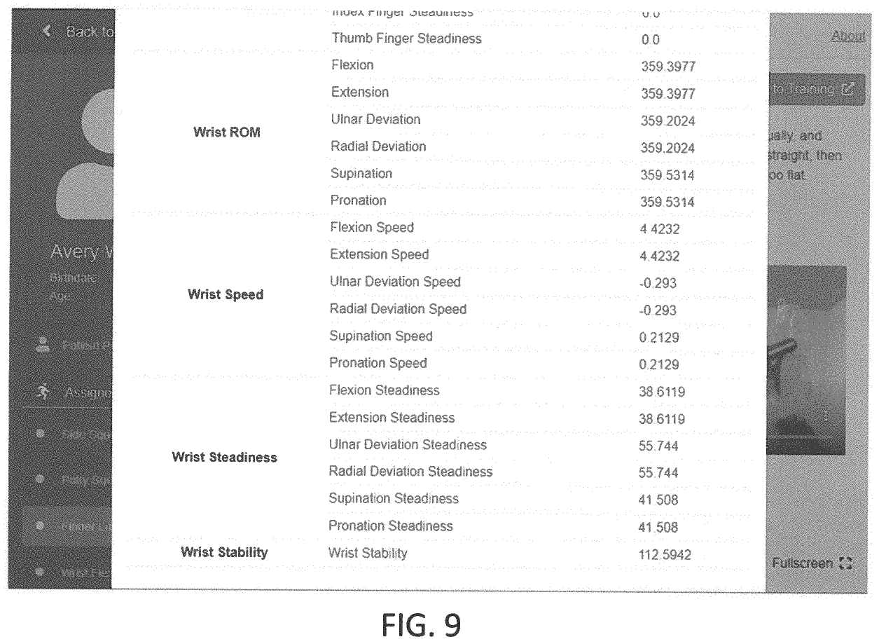

The patient can interact with their computing device 13 which includes suitable software thereon to enable the patient to view the information prepared by the therapist such as the schedule of FIG. 6 and the demonstration videos and supplementary information according to FIG. 5. When the patient initiates a scheduled exercise, the system will capture all relevant sensor data and optionally may also use the camera of the computing device 13 to capture video images relating to the user performing the exercise. Upon authorization by the patient user, the video and all sensor data can be returned to the remote server for processing and/or analysis, or returned to the computer device of the therapist for processing and/or analysis. The processing of the sensor data can include calculation of various performance metrics representative of the sensed operating characteristics from the movement sensors during the exercise to generate a graph illustrating a variation of the performance metric or operating characteristic over a duration of the exercise by the user for display on a display of the user computing device or subsequent display to the therapist. Exemplary graphs are represented in FIG. 8. The system can also generate a video comprising images graphically representing the calculated position of the finger sections and the hand section throughout the exercise by the user and also graphically represent the position of each finger as it is flexed from a starting position to an ending position as represented schematically in FIG. 4. The system can further record all operating characteristics and calculate relevant performance metrics for display in a chart to both the patient or the therapist as represented in FIG. 9.

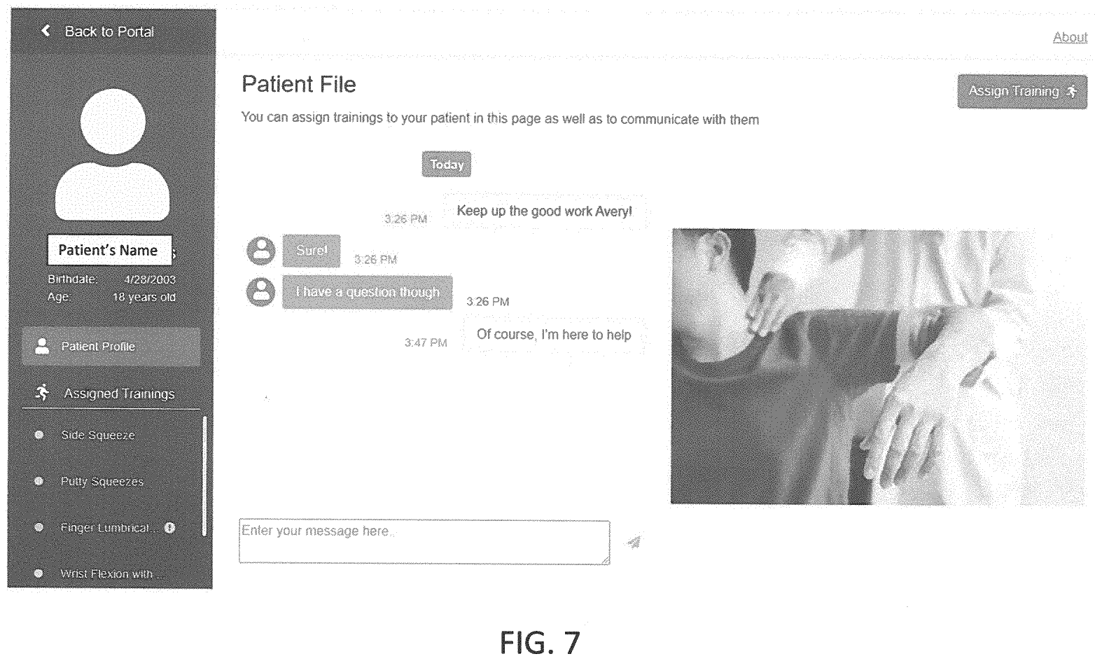

The software can be further enabled with functionality for a chat function, as represented in FIG. 7, or videoconferencing between the patient computing device 13 and the therapist computer device 21.

Since various modifications can be made in the invention as herein above described, and many apparently widely different embodiments of same made, it is intended that all matter contained in the accompanying specification shall be interpreted as illustrative only and not in a limiting sense.

1. A rehabilitation system for measuring performance of an upper limb rehabilitation exercise of a user relative to a target movement using a computing device, the system comprising:
(i) a rehabilitation apparatus comprising:
a hand section arranged to be secured relative to a backside of a hand of the user;
at least one hand movement sensor operatively connected to the hand section so as to be arranged to sense one or more operating characteristics of the hand section;
a plurality of finger sections arranged to be secured relative to respective fingers of the user and being movable relative to the hand section such that the fingers sections are movable with the fingers of the user relative to the backside of the hand of the user;
finger movement sensors operatively connected to the finger sections so as to be arranged to sense one or more operating characteristics of the finger sections; and
a sensor interface in communication with the at least one hand movement sensor and the finger movement sensors; and
(ii) a computer readable memory storing programming instructions thereon which are executable by the computing device such that the computing device is arranged to:
communicate with the sensor interface to obtain the sensed operating characteristics from the movement sensors during the upper limb rehabilitation exercise by the user; and
compare the sensed operating characteristics from the movement sensors to corresponding stored operating characteristics representative of the target movement.

2. The system according to claim 1 wherein the programming instructions on the computer readable memory are further arranged to be executed by the computing device such that the computing device is arranged to acquire the stored operating characteristics representative of the target movement from a previous upper limb exercise executed on the rehabilitation apparatus.

3. The system according to claim 1 wherein the programming instructions on the computer readable memory are further arranged to be executed by the computing device such that the computing device is arranged to acquire the stored operating characteristics representative of the target movement from a remote computer device in communication with the computing device over a communications network.

4. The system according to claim 1 further comprising an auxiliary apparatus which is symmetrical in configuration to the rehabilitation apparatus such that one of the apparatuses is arranged to be worn on a right hand of the user and another of the apparatuses is arranged to be worn on a left hand of the user, each of the auxiliary apparatus and the rehabilitation apparatus having said at least one hand movement sensor, said finger movement sensors and said sensor interface with which the computing device is arranged to communicate when executing the programming instructions stored on the computer readable memory, the programming instructions on the computer readable memory being further arranged to be executed by the computing device such that the computing device is arranged to (i) communicate with the sensor interface of the auxiliary apparatus to acquire said stored operating characteristics representative of the target movement from a previous upper limb exercise executed on the auxiliary apparatus, (ii) communicate with the sensor interface of the rehabilitation apparatus to obtain the sensed operating characteristics from the movement sensors during the upper limb rehabilitation exercise by the user operating the rehabilitation apparatus, and (iii) compare the sensed operating characteristics from the rehabilitation apparatus to the stored operating characteristics from the auxiliary apparatus.

5. The system according to claim 1 wherein the comparison of the sensed operating characteristics from the movement sensors to corresponding stored operating characteristics representative of the target movement includes (i) a calculation of at least one performance metric representative of the sensed operating characteristics from the movement sensors during the upper limb rehabilitation exercise by the user and (ii) a calculation of a similarity value comprising a measure of similarity between said at least one performance metric and a corresponding performance metric defined by the stored operating characteristics representative of the target movement.

6. The system according to claim 1 further comprising:
the stored operating characteristics defining a range of motion of the target movement; and
the programming instructions on the computer readable memory being further arranged to be executed by the computing device such that the computing device is arranged to calculate a range of motion relating to the upper limb rehabilitation exercise from the sensed operating characteristics from the movement sensors during the upper limb rehabilitation exercise and compare the calculated range of motion to the range of motion defined by the stored operating characteristics.

7. The system according to claim 1 further comprising:
the movement sensors including angular velocity sensors;
the stored operating characteristics defining a speed of motion of the target movement; and
the programming instructions on the computer readable memory being further arranged to be executed by the computing device such that the computing device is arranged to calculate a speed of motion relating to the upper limb rehabilitation exercise from the sensed operating characteristics from the angular velocity sensors during the upper limb rehabilitation exercise and compare the calculated speed of motion to the speed of motion defined by the stored operating characteristics.

8. The system according to claim 1 further comprising:
the movement sensors including angular acceleration sensors;
the stored operating characteristics defining a motion steadiness of the target movement; and
the programming instructions on the computer readable memory being further arranged to be executed by the computing device such that the computing device is arranged to calculate a motion steadiness relating to the upper limb rehabilitation exercise from the sensed operating characteristics from the angular acceleration sensors during the upper limb rehabilitation exercise and compare the calculated motion steadiness to the motion steadiness defined by the stored operating characteristics.

9. The system according to claim 1 further comprising:
the movement sensors including linear acceleration sensors;
the stored operating characteristics defining a motion stability of the target movement; and
the programming instructions on the computer readable memory being further arranged to be executed by the computing device such that the computing device is arranged to calculate a motion stability relating to the upper limb rehabilitation exercise by calculating a derivative of an acceleration acquired from the sensed operating characteristics from the linear acceleration sensors during the upper limb rehabilitation exercise and compare the calculated motion stability to the motion stability defined by the stored operating characteristics.

10. The system according to claim 1 wherein the programming instructions on the computer readable memory are further arranged to be executed by the computing device such that the computing device is arranged to calculate at least one performance metric from the sensed operating characteristics from the movement sensors during the upper limb rehabilitation exercise by the user such that said at least one performance metric defines a movement of the fingers of the user.

11. The system according to claim 1 wherein the programming instructions on the computer readable memory are further arranged to be executed by the computing device such that the computing device is arranged to calculate at least one performance metric from the sensed operating characteristics from the movement sensors during the upper limb rehabilitation exercise by the user such that said at least one performance metric defines a movement of the hand of the user.

12. The system according to claim 1 wherein the programming instructions on the computer readable memory being further arranged to be executed by the computing device such that the computing device is arranged to calculate at least one performance metric from the sensed operating characteristics from the movement sensors during the upper limb rehabilitation exercise by the user such that said at least one performance metric defines a movement of the wrist of the user.

13. The system according to claim 1 wherein the programming instructions on the computer readable memory are further arranged to be executed by the computing device such that the computing device is arranged to calculate a performance metric representative of the sensed operating characteristics from the movement sensors during the upper limb rehabilitation exercise by the user and generate a graph illustrating a variation of the performance metric over a duration of the upper limb rehabilitation exercise by the user for display on a display of the computing device.

14. The system according to claim 1 wherein the programming instructions on the computer readable memory are further arranged to be executed by the computing device such that the computing device is arranged to (i) calculate a position of the finger sections and the hand section throughout the upper limb rehabilitation exercise by the user, and (ii) generate a video comprising images graphically representing the calculated position of the finger sections and the hand section throughout the upper limb rehabilitation exercise by the user.

15. The system according to claim 1 wherein the programming instructions on the computer readable memory are further arranged to be executed by the computing device such that the computing device is arranged to (i) receive video images from a remote computer device over a communications network and display the video images on a display of the computing device, and (ii) capture video images of the user using a camera of the computing device and communicate the captured video images with the sensed operating characteristics to the remote computer device over the communications network.

16. The system according to claim 1 further comprising a plurality of resilient finger members, each resilient finger member being arranged to be operatively connected between the hand section and a respective one of the finger sections so as to be flexed with movement of the respective finger section relative to the hand section, at least some of the resilient finger members having a different stiffness relative to one another and being interchangeable with one another to vary a resistance of flexing movement of the finger sections.

17. The system according to claim 1 further comprising:
the rehabilitation apparatus further comprising a wrist section arranged to be secured relative to a forearm of the user, the system may further include a plurality of resilient wrist members, each resilient wrist member being arranged to be operatively connected between the hand section and the wrist section so as to be flexed with movement of the wrist section relative to the hand section, the resilient wrist members having a different stiffness relative to one another and being interchangeable with one another to vary a resistance of movement of the hand section relative to the wrist section.

18. The system according to claim 1 wherein the rehabilitation apparatus further comprises:
a glove including a hand portion arranged to extend over the backside of the hand of the user and a plurality of finger portions arranged to be worn on the fingers the user;
a plurality of first connectors arranged to releasably connect the finger portions of the glove to the finger sections of the rehabilitation apparatus; and
one or more second connectors arranged to releasably support the hand section relative to the hand portion of the glove when the glove is worn on the hand of the user;
the glove being readily separable from the hand section and the finger sections of the rehabilitation apparatus so as to be interchangeable with another glove of identical configuration.

19. The system according to claim 1 wherein the rehabilitation apparatus further comprises one or more vital sign monitoring sensors supported on the apparatus for communication with the user so as to be arranged to sense a state of one or more essential body functions of the user, the programming instructions on the computer readable memory being further arranged to be executed by the computing device such that the computing device is arranged to compare the sensed state to a threshold to determine a notification condition.

20. A method of rehabilitation using the system of claim 1, the method comprising:
supporting the rehabilitation apparatus on the hand of the user; and
performing the upper limb rehabilitation exercise including any one of or all of bending, pinching, flexion, extension, adduction and abduction of the fingers or bending, flexion, extension, adduction and abduction of the wrist.
